(19) United States
(12) Patent Application Publication
(10) Pub. No.: US 2023/0301600 A1
(43) Pub. Date: Sep. 28, 2023

(54) METHOD FOR DETERMINING A LEVEL OF CERTAINTY OF A PATIENT'S RESPONSE TO A STIMULUS PERCEPTION OF A SUBJECTIVE MEDICAL TEST AND A DEVICE THEREFORE

(71) Applicant: Essilor International, Charenton-Le-Pont (FR)

(72) Inventors: Hélène STARYNKEVITCH, Versailles (FR); Vaïa MACHAIRAS, Saint-Maur-des-Fosses (FR)

(73) Assignee: Essilor International, Charenton-Le-Pont (FR)

(21) Appl. No.: 18/065,249

(22) Filed: Dec. 13, 2022

(30) Foreign Application Priority Data
Dec. 13, 2021 (EP) 21306756.4

(51) Int. Cl. A61B 5/00; A61B 3/02

(52) U.S. Cl. CPC A61B 5/7267; A61B 3/02

(57) ABSTRACT

A computer implemented method for determining a level of certainty of a patient’s response to a stimulus perception of a subjective medical test, the method including detecting at least one physiological signal from the patient while the patient is providing a response to the stimulus perception, determining the level of certainty of a patient’s response to the stimulus perception from the at least one physiological signal, the at least one physiological signal being an input data to a machine learning model trained based on a set of training data, the set of training data comprising at least one physiological signal associated to a level of certainty of a patient’s response, the determined level of certainty being the output of the trained machine learning model.

### FIELD OF THE INVENTION

Computer implemented method for determining a level of certainty of a patient’s response to a stimulus perception of a subjective medical test and a device therefore, in particular a subjective ophthalmic test.

### BACKGROUND OF THE INVENTION

Usually, while performing a subjective test such as subjective refraction, the practitioner should take into account different parameters (objective and subjective) to choose the next “ stimulus perception ”, i.e. to choose next stimulus or next lens. One of the main subjective parameters is the response of the patient to the stimulus, in other words, it’s level of sensitivity to the stimulus: (“yes” / “no” / “EABCD” / “1” / “2” / ....). The practitioner values the certainty of this answer to check if this answer is correct (indeed it’s seen by the patient) or if it’s more guessed. The appreciation of the patient’s certainty will help the practitioner to ponder the content of the response in order to make a decision or to adjust the next step of the subjective test (or final evaluation) with regards to the patient case. This adjustment is performed on the basis of the practitioner’s skills, firstly according to his own appreciation of the patient’s response and the patient’s certainty, and secondly according to his practice knowledge and his experience to decide and to adapt the next step (or final evaluation).

For example, EP3272274 describes a method for measuring the dioptric parameters of a person by taking account the degree of certainty of the person upon expressing the visual assessment. In this application, the degree of certainty may be evaluated by the practitioner.

However, it is known that the practitioner appreciation of the patient’s certainty and resulting adaptation of the next step is one of the reasons explaining the high practitioners’ variability on refraction results for the same set of patients, and hence the quality of refraction results.

Moreover, when subjective tests are automated, the model used for the evaluation of the patient’s certainty is not personalized. Indeed, according to the patient, the level of certainty obtained may not be well suited for the patient (less accurate value, ...).

Therefore, there is a need for a method that would allow an automated measurement of the patient’s certainty in order to improve subjective test reproducibility among practitioners.

### SUMMARY OF THE INVENTION

The invention is defined by the appended independent claims. Additional features and advantages of the concepts herein disclosed are set forth in the description which follows.

The present disclosure aims at improving the situation. In particular, one aim of the invention is to overcome the above mentioned drawbacks.

To this end, the present disclosure describes a computer implemented method for determining a level of certainty of a patient’s response to a stimulus perception of a subjective medical test, the method comprising:
-detecting at least one physiological signal from the patient (15) while the patient is providing a response to the stimulus perception,
-determining the level of certainty of the patient’s response to the stimulus perception (16) from the at least one physiological signal (15),
   the at least one physiological signal (15) being an input data to a machine learning model (10) trained based on a set of training data,
   the set of training data comprising at least one physiological signal (11) associated to a level of certainty (12) of a patient’s response,
   the determined level of certainty being an output of the trained machine learning model.

The level of certainty may comprise three different notions:

“Certainty”: final choice of one option by eliminating the others. Such as “Yes/No”.

“Doubt”: critical questioning of the various options which make it possible to choose the most probable but without totally excluding the others. Such as “May be...”.

“Hesitation”: inability to choose between various options. Such as “I don’t know”.

These 3 notions may be converted into several levels of certainty. Moreover, the “doubt” level could also be subdivided into several intermediate levels to have a finer mesh.

It’s clear from the present disclosure (above and below) that the output of the trained machine learning model is the level of certainty of the patient’s response to a stimulus perception of a subjective medical test. and not the result of the subjective medical test.

By “subjective medical test”, we mean at least one stage for which the patient needs to communicate its sensitivity to a stimulus perception when the response of the patient is an important criterion to obtain the result of the subjective medical test. The subjective medical test may comprise at least one stage. The subjective medical test may be a sub-part of a medical test.

By “response”, we mean for example answering a question, speaking, writing, clicking, choosing an option in order to express his level of sensitivity to a stimulus perception.

By “stimulus perception”, we mean a perception of a stimulus such as a visual stimulus (light, picture, ....), an auditory stimulus, an olfactory stimulus, a touching stimulus, a sensitive stimulus in a specific condition. The stimulus perception may comprise an origin of the stimulus and a corrective element of the origin of the stimulus. For example, for the ophthalmic application, the stimulus perception may comprise an optotype (origin of the stimulus) and a lens through which the patient watches the picture (correction of the origin of the stimulus).

Detecting at least one physiological signal from the patient (15) may be made while the stimulus perception is provided to the patient during a subjective medical test and the patient is providing a response to the stimulus perception.

Detecting at least one physiological signal from the patient (15) may be made while the patient is providing a response to the stimulus perception.

This method allows assessing the level of certainty of a patient’s response in an objective way in order to simplify the subjective medical test and to obtain better results than with an usual subjective medical test when the response of the patient is an important criteria to obtain the result of the test. Indeed, thanks to this method, the variability of the results of the stage linked to the patient’s certainty appreciation performed by practitioners is removed.

Thus, the inventors have shown that thanks to this method it is possible to obtain an automated or semi-automated measurement of the patient’s certainty in order to improve subjective reproducibility of a subjective medical test among practitioners by removing the variability linked to the patient’s certainty appreciation performed by practitioners.

According to further embodiments which can be considered alone or in combination, the method may comprise further a step of inter and / or intra personal homogenizing the input or the output of the trained machine learning.

By inter personal homogenizing, we mean taking into account the variability between each patient.

By intra personal homogenizing, we mean taking into account the variability of one patient at different time.

By variability, we mean the range of possible values for any characteristic, physical or mental, of human being such as gender, age, cognitive ability, personality, mood, fatigue, refraction value ......

Thanks to this embodiment, the variability linked to the difference between each patient is removed. Thus, the subjective medical test is simplified, more efficient, more reproducible whatever the characteristic of the patient.

The step of inter and/or intra personal homogenization may comprise the step of standardizing the at least one physiological signal, the at least one standardized physiological signal being the input data to the trained machine learning.

By standardizing, we mean putting different variables on the same scale, in other words, a scaling technique where the values of the at least one physiological signal are centered around a mean with a low standard deviation (such as 1 or a unit standard deviation). This means that the mean of the attribute becomes zero and the resultant distribution has a unit standard deviation.

This embodiment may be a way to take into account the variability between different patient or between different time of a same patient.

According to further embodiments which can be considered alone or in combination, the step of inter and/or intra personal homogenizing may comprise a step of detecting at least one reference physiological signal associated to a reference level of certainty of the patient’s response. The at least one reference physiological signal and the reference level of certainty of the patient’s response are a set of reference data. The level of certainty of the patient’s response to the stimulus perception is determined from the at least one physiological signal and from the set of reference data.

This embodiment presents the advantage to personalize the determination of the level of certainty according to the patient and his features at the moment of the test or between different patient. Further, it allows enriching the input data to improve the training of the machine learning model.

This embodiment may be a way to take into account the variability between different patient or between different time of a same patient.

The reference level of certainty of the patient’s response may be used as an input to the trained machine learning model. This embodiment presents the advantage to obtain an output of the training machine model directly relevant and interpretable.

The reference level of certainty of the patient’s response may be used to threshold the output data. This embodiment presents the advantage to use directly the input and make the output of the training machine model relevant and interpretable.

According to further embodiments which can be considered alone or in combination, the physiological signals comprise signals having different modalities and the method comprises formatting the physiological signals having different modalities. Thus, it is possible to use different kinds of physiological signals such as video, audio, text along with micro-expression, pressure measurement, temperature measurement....

According to further embodiments which can be considered alone or in combination, the level of certainty is a category. The step of determining the category of certainty comprises classifying the input data by means of the trained machine learning model to determine the level of certainty. This embodiment with the category make the output easy and quick to interpret.

According to further embodiments which can be considered alone or in combination, the level of certainty is a score. The step of determining the score of certainty comprises regressing the input data by means of the trained machine learning model to determine the level of certainty. This embodiment makes the output accurate.

According to further embodiments which can be considered alone or in combination, the output and/or the input data is post processed, such as by normalizing.

The subjective test may be an ophthalmic test with a visual stimulus perception, for example a refraction test (spherical cylinder, astigmatism), an assessing of the sensitivity to the light, Cross cylinder test, red/green test, binocular test, defog test, an assessing of the dominant eye, binocular equilibrium test, addition test. The subjective test may be an audio test with an audio stimulus perception or any kind of medical subjective test, or an olfactory test or a touching test or a pain test.

Further, the present disclosure describes a method for a subjective medical test, which comprises
determining the level of certainty according to the present disclosure, and
informing of the determined level of certainty, and/or
weighting a result of the subjective medical test, and/or
changing manually or automatically the stimulus perception according to the determined level of certainty.

The level of certainty may be considered as a weight to appreciate the result of a stage of the subjective medical test and/or a weight to choose the next relevant stage of the medical subjective test to be perform in the subjective medical test.

The stage of the subjective medical test comprises a stimulus perception and a patient’s response to the stimulus perception. The result of the stage is the appreciation of the patient’s response.

Further, the present disclosure describes a device for a subjective medical test of a patient, comprising:
a control unit configured to determine the level of certainty of a patient’s response to a stimulus perception of the subjective medical test
the level of certainty being determining from at least one physiological signal of the patient while the patient is providing the response to the stimulus perception,
the at least one physiological signal being as an input data to a trained machine learning model,
the determined level of certainty being an output of the trained machine learning model.

The control unit is configured to determine the level of certainty of the patient’s response to the stimulus perception from the at least one physiological signal according to the method of the present disclosure and according to the embodiment of the method of the present disclosure.

The control unit may be located at the same place than the one of the subjective medical test or at a different place than the one subjective medical test.

The control unit may be located at the same place than the one of the patient or at a different place than the one of the patient.

It presents the advantage to be used for telemedicine / tele optometry or to be used in face to face with the practitioner.

According to further embodiments which can be considered alone or in combination, the device comprises further
a test unit configured to provide a subjective test associated to stimulus perceptions, and
a detector configured to detect at least one physiological signal from the patient while the patient is providing a response to the stimulus perception.

The detector may be at least one microphone and/or at least one camera and/ or at least one pressure detector (pressure on an object or blood pressure) and/or at least one temperature detector, electroencephalogram, electroretinogram, cardiac frequency detector or any detector which can measure a physiological signal of the patient.

The subjective medical test of the medical device may be an ophthalmic test associated to a stimulus perception such as a visual stimulus perception.

Embodiments discussed herein are merely representative and do not limit the scope of the invention. It will also be obvious to one skilled in the art that all the technical features that are defined relative to a process can be transposed, individually or in combination, to a device and conversely, all the technical features relative to a device can be transposed, individually or in combination, to a process.

### BRIEF DESCRIPTION OF THE DRAWINGS

Embodiments of the invention will now be described, by way of example only, and with reference to the following drawings in which:

- FIG. 1 is a flow chart representing the method for determining a level of certainty of a patient’s response to a stimulus perception of a subjective medical test according to an embodiment of the present description,

- FIGS. 2A and 2B are two flow charts representing a method for determining a level of certainty of a patient’s response to a stimulus perception of a subjective medical test according to two examples of embodiments of the present description,

- FIG. 3 is a flow chart representing a method for determining a level of certainty of a patient’s response to a stimulus perception of a subjective medical test according to one example of an embodiment of the present description,

- FIGS. 4A and 4B are two flow charts representing a method for determining a level of certainty of a patient’s response to a stimulus perception of a subjective medical test according to three examples of embodiments of the present description,

- FIG. 5 illustrates a device according to an embodiment of the invention.

### DETAILED DESCRIPTION OF PREFERRED EMBODIMENTS

In the description which follows the drawing figures are not necessarily to scale and certain features may be shown in generalized or schematic form in the interest of clarity and conciseness or for informational purposes. In addition, although making and using various embodiments are discussed in detail below, it should be appreciated that as described herein are provided many inventive concepts that may be embodied in a wide variety of contexts.

Embodiments discussed herein are merely representative and do not limit the scope of the invention. It will also be obvious to one skilled in the art that all the technical features that are defined relative to a process can be transposed, individually or in combination, to a device and conversely, all the technical features relative to a device can be transposed, individually or in combination, to a process.

To avoid unnecessary details for practicing the invention, the description may omit certain information already known to those skilled in the art.

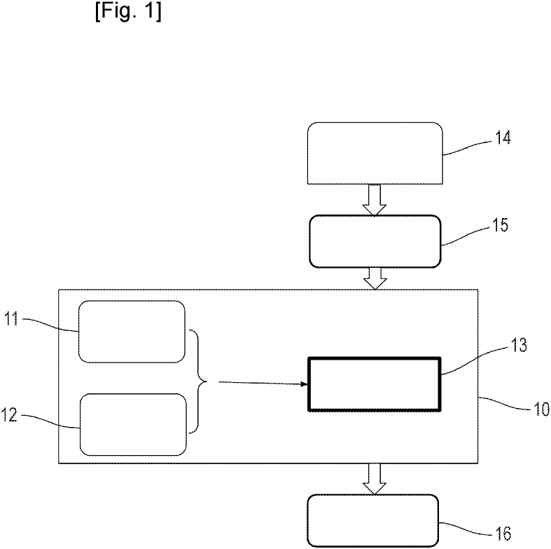

FIG. 1 illustrates a flow chart representing a method for determining a level of certainty of a patient’s response to a stimulus perception of a subjective medical test according to an embodiment of the present description.

A subjective medical test may be at least one stage for which the patient needs to communicate its stimulus perception when the response of the patient is an important criterion to obtain the result of the subjective medical test.During a medical subjective test, a stimulus perception is presented to a patient.

The stage of the subjective medical test comprises a stimulus perception and a patient’s response to the stimulus perception. The result of the stage is the appreciation of the patient’s response. The subjective medical test may comprise at least one stage. The subjective medical test may be a sub-part of a medical test.

The subjective medical test may be an ophthalmic test with a visual stimulus perception (optometry chart, light, color...) for example a refraction test (spherical cylinder, astigmatism) such as cross cylinder test, red/green test, binocular test, defog test, balance test, addition test; an assessing of the sensitivity to the light, an assessing of the dominant eye, binocular test. The subjective medical test may be the entire medical test (e.g. determination of the correction), part of the medical test (e.g. determination of the OD sphere), or a sub-part of the subjective medical test (e.g. a step in the determination of the OD sphere).

The subjective medical test may be an audio test with an audio stimulus perception or any kind of medical subjective medical test.

The subjective medical test may be a patient pain evaluation or a patient comfort evaluation. The subjective medical test may be realized in a medical center (ophthalmologists, hospital, ...) or in shop (optometry, glasses shop, ...), or in remote such as telemedicine/teleoptometry or in research center.

The subjective medical test may be made by a practitioner or by the patient himself or any kind of person or by a device.

The stimuli perception is provided to the patient. By “stimulus perception”, we mean a perception of a stimulus such as a visual stimulus (light, picture, ....), an auditory stimulus, an olfactory stimulus, a touching stimulus, a sensitive stimulus in a specific condition. The stimulus perception may comprise an origin of the stimulus and a corrective element of the origin of the stimulus. For example, for the ophthalmic application, the stimulus perception may comprise an optotype (origin of the stimulus) and a lens through which the patient watches the picture (correction of the origin of the stimulus).

The patient is providing a response to the stimulus perception. By “response”, we mean for example answering a question, speaking, writing, clicking, choosing an option in order to express his level of sensitivity to a stimulus perception. For example, by answering to a question with different options via a practitioner or a computer such as “do you see the letter?”, “Is it better now?”, “do you heard a sound?”)

The patient chooses one option among the presented options with a level of certainty. The level of certainty may comprise three different notions:

“Certainty”: final choice of one option by eliminating the others. Such as “Yes/No”.

“Doubt”: critical questioning of the various options which make it possible to choose the most probable but without totally excluding the others. Such as “May be...”.

“Hesitation”: inability to choose between various options. Such as “I don’t know”.

These 3 notions may be converted into several levels of certainty. Moreover, the “doubt” level could also be subdivided into several intermediate levels to have a finer mesh.

The difference between “hesitation” and “doubt” lies in the patient’s final answer: does he manage to make a choice or not?

As illustrated FIG. 1, the method comprises the step of providing a set of training data. The set of training data comprises at least one physiological signal 11 associated to a level of certainty 12 of a patient’s response.

The at least one physiological signal may be physiological data (blood pressure, sweat,...), face expression, body expression, voice and sounds analysis and emotion, time to answer, communicate, any other pertinent quantity to determine the patient behaviour/reaction, patient personal data: age, gender, ...

Some examples of specific physiological data are given below:
Voice :
   Tone of voice (relative decibels)
   Duration of each word
   Breathing & sighs
   Silences
Language:
   Onomatopoeias of doubt / hesitation: “uh”, “mmm...”,...
   Semantics of doubt / hesitation: “not sure”,...
   Semantics of the expression of a personal opinion: “I think”, “it seems to me”, “maybe”
Body:
   Gaze direction
   Furrowed eyebrows, raised eyebrows
   Position discomfort (the patient wiggles, etc.)
   Physiological signals: heart rate, perspiration, respiratory rate, blood pressure and even pupillary diameter, ...

For example, for a high level of “Certainty”, we may use for the set of training data:
The answer is given in a confident tone.
Response time for example <3 seconds, for example <5 seconds.

For example, for “Hesitation” and “Doubt”, we may use for the set of training data:
Response time for example ≥ 5 seconds
Number of times the patient wants to review the different options.

Thus, obtaining the set of training data may comprise:
Determining the panel of patients: Choosing ages, genders, country, with or without pathologies, different visual corrections (sphere, cylinder, axis, addition, strabismus, ...);
Determining the subjective medical test exam (and the conditions in which it is realized for example in remote or in a hospital);
Recording the physiological signals (voice, signals, etc.) as input to each patient’s response during his examination. Calibration / calibration specific to sensor may be required;
Recording the level of certainty associated with these inputs for each response from the patient during his test.

According to an embodiment, it may comprise:
Recording reference physiological signal. For example, at least one question to ask to the patient (to take all the reference physiological signal and the associated level of certainty). Example of questions:
   “What is your name ?” → certainty
   “What year is it ?” → certainty
   “Do you prefer blue or pink?” → doubt or hesitation or certainty
   “Do you prefer jazz or blues?” → doubt or hesitation or certainty
   “What is the 10th decimal place of Pi?” → hesitation

An example of embodiment to determine the level of certainty associated to the reference physiological signal may be realized/assessed by a human such as the patient or the practitioner or the practitioner or the patient or multiple practitioners/patients.

Alone or in combination, it may help to determine the level of certainty associated to the reference physiological signal by establishing decision rules which will allow the practitioner to choose the level of certainty as reproducibly as possible. The rules may be based on the example of paragraphs [079] to [086].

In addition, to reduce the inter-staff bias of this assessment, several practitioners may be asked to be present in the room to also assess the level of certainty (in addition to the practitioner who performs the examination and who marks him).

Then, the set of training data 11, 12 is used to train a machine learning model 13.

### Machine Learnin Model Training

The set of training data may be used to train the machine learning model. These data may be in the same format and contain the same information as what will be provided to the trained machine learning model later to make a prediction.The machine learning model may take as input a training set of observed data points to “learn” an equation, a set of rules, or some other data structure. This learned structure or statistical model may then be used to make generalizations about the training set or predictions about new data. As used herein, “statistical model” refers to any learned and/or statistical data structure that establishes or predicts a relationship between two or more data parameters (e.g., inputs and outputs). Although the invention is described below with reference to neural networks, other types of statistical models may be employed in accordance with the present invention.

For example, data point of the set of the training data may include a set of values that is linked with, or predict, another value in the data point. In the present invention, the machine learning model is configured to link at least one physiological signal related to a level of certainty provided to the machine learning model as inputs to a behaviour of the patient.

Training of the machine learning model may be performed by providing the model with a plurality of initial data related for example to a set of initial patient as explained before.

Said set of training data comprise a plurality of acquired learning signals representative of a variation of at least one characteristic of at least physiological signal related to a level of certainty for each initial patients of the set.

This training is performed iteratively until the model is accurate enough. As an example, training the model may imply at least one hundred initial patients.

The input data may be chosen specifically according to a given subjective medical test.

Thus the machine learning model is a trained machine learning model.

The training of the machine learning model may be done on a different computer/control unit than the one used for determining the level of certainty based on the trained machine learning model, or on the same computer/control unit.

The training of the machine learning model may be done at the same time or at a different time than the time when the level of certainty is determined based on the trained machine learning model.

The training of the machine learning model may be done in one shot or several shots and/or upgraded regularly or at each using.

### Machine Learnin Model Architecture

Said machine learning model may be based either on a long short-term memory (LSTM / for a text document) technique or a convolutional neural network (CNN / for a picture).

In particular, according to the input data, different types of model may be used, for example
The vocal answer may be transformed into text thanks to a speech-to-text model. Then, the text may be analysed with a natural language understanding model (ex: RNN including LSTM),
The images, in particular the images of the patient of the patient can be processed with a CNN,
For example, all processed signals (with specific neural networks) may be joined as input to the “last part ” neural network whose output is the level of certainty of the patient.

LSTM technique is part of recurrent neural networks (RNNs). Classical RNNs techniques comprise a network of neural nodes organized in successive layers. Each node (neuron) in a given layer is connected one-way to each of the nodes in the next layer. This structure allows previous moments to be taken into account in the neural network, since a first layer for a former moment t-1 is connected to second layer for a moment t. This second layer is also connected to a third layer for a subsequent moment t+1, and so on with a plurality of layers. Each signal provided as an input is therefore processed in a temporal way, taking into account the signals provided at former moments.

CNN techniques use the signals as images, not in a temporal way. The plurality of acquired signals is processed at once with all the data acquired for a test duration. Mathematical image processing operations are then applied to the image obtained with the plurality of acquired signals, e.g. convolution integral, to determine outputs of the machine learning model. CNN may comprise different layers as convolution layers, pooling layer (max pooling), batch normalization, activation....

As illustrated in FIG. 1, the next step is to provide a subjective medical test 14 associated to stimulus perceptions.

As illustrated in FIG. 1, the next step is to provide a subjective medical test 14 associated to a stimulus perception.

As explained before, by providing the response to the stimulus perception, we mean expressing the level of sensitivity to the stimulus perception by answering to a question with different options via a practitioner or a computer. It could be for example, answering to a question when the stimulus perception is presented to the patient, such as “do you see the stimulus perception?”, “which letter can you read? », « do you heard the sound? », «is it better with the lens 1 or 2?», “do you see difference between 1 and 2 ?”,....

The patient could provide the response directly to a practitioner or via a numerical interface (screen, tablet, smartphone, computer or a mic or a speech recognition device).

The at least one physiological signal may be physiological data (blood pressure, sweat, ...), face expression, body expression, voice and sounds analysis and emotion, time to answer/communicate/provide, any other pertinent quantity to determine the patient behaviour/reaction, patient personal data: age, gender, nationality, country of birth, country of living...

The nature of input data may be: images, sounds, physiological signals, ...

As illustrated in FIG. 1, the next step is to determine the level of certainty of the patient’s response to the stimulus perception 16 from the at least one physiological signal 15 as an input data to the trained machine learning model 13. The determined level of certainty of the patient’s response to the stimulus perception is the output of the trained machine learning model.

The output of the trained machine learning model is not the result of the subjective medical test. The output of the trained machine learning model is the level of certainty of the patient’s response to a stimulus perception of a subjective medical test. The level of certainty may be considered as a weight to appreciate the result of a stage of the subjective medical test and/or a weight to choose the next relevant stage to be perform in the subjective medical test.

The at least one physiological signal may be used directly as input data or may be preprocessed such as removing the noise or normalizing.

The output of the model could be a score between 0 and 1 giving the level of certainty of the patient for a given answer (0: uncertain, 1 : certain), or an equivalent classification into classes of certainty.

According to further embodiments which can be considered alone or in combination, the level of certainty is a category. The step of determining the category of certainty comprises classifying the input data by means of the trained machine learning model to determine the level of certainty.

According to further embodiments which can be considered alone or in combination, the level of certainty is a score. The step of determining the score of certainty comprises regressing the input data by means of the trained machine learning model to determine the level of certainty.

The way to establish the model could be (but is not limited to) supervised learning.

Determination of level of certainty from new physiological signals is predicted by applying the training machine learning model.

This method allows to assess the level of certainty of a patient in an objective way in order to simplify the subjective medical test and to obtain better results with an usual subjective medical test. Indeed, thanks to this method, the variability linked to the patient’s certainty appreciation performed by practitioners is removed.

Thus, the inventors have shown that thanks to this method it is possible to obtain an automated or semi-automated measurement of the patient’s certainty in order to improve subjective reproducibility of a subjective medical test among practitioners (removing the variability linked to the patient’s certainty appreciation performed by practitioners).

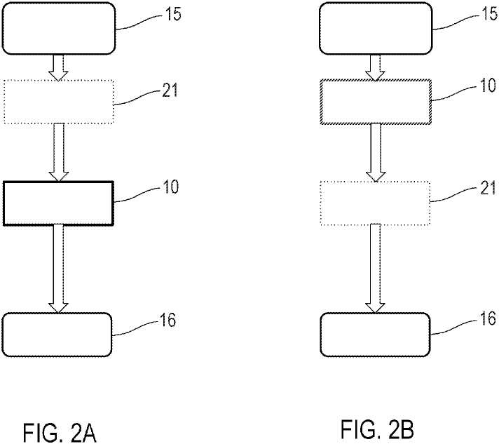

The FIGS. 2A and 2B are two flow charts representing an embodiment of the method for determining a level of certainty of a patient’s response to a stimulus perception of a subjective medical test according to the present description.

As in FIG. 1, the references 15, 10, 16 are the same. The steps and elements 11, 12, 13, 14 are not shown in FIGS. 2A and 2B but they may be comprised in the embodiment of FIGS. 2A and 2B.

According to the embodiment of FIGS. 2A and 2B, the method may comprise a step of inter and / or intra personal homogenizing 21 the input data or the output of the trained machine learning.

By inter personal homogenizing, we mean taking into account the variability between different patient.

The inputs may be inter personal homogenized. Thus before to provide the input to the trained machine learning model, the inputs are homogenized, for example the physiological signals.

The ouput may be inter personal homogenized such as the level of certainty are homogenized.

By intra personal homogenizing, we mean taking into account the variability of one patient at different time. By normalizing, we mean taking into account the variability between each patient.

The inputs may be intra personal homogenized. Thus before to provide the input to the trained machine learning model, the inputs are homogenized, for example the physiological signals.

The ouput may be intra personal homogenized such as the level of certainty are homogenized.

All the combinations, in terms of input /output and/or intra/inter of homogenizing are possible.

By variability, we mean the range of possible values for any characteristic, physical or mental, of human beings such as gender, age, cognitive ability, personality, mood, fatigue, refraction value......

Thanks to this embodiment, the variability linked to the difference and the specificity of emotion between each patient is removed or at least taken into account.

Thus, the subjective medical test is simplified, more efficient, more reproducible whatever the characteristic of the patient.

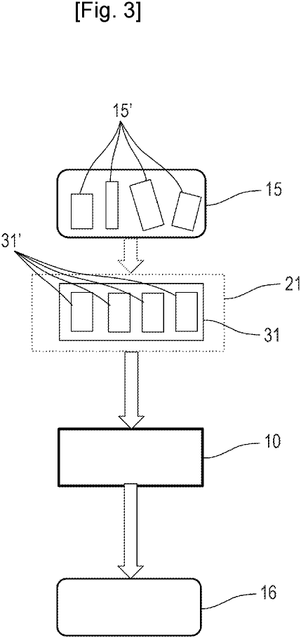

The FIG. 3 is a flow chart representing an embodiment of the method for determining a level of certainty of a patient’s response to a stimulus perception of a subjective medical test according to the present description.

As in FIG. 1, the references 15, 10, 16 are the same. The steps and elements 11, 12, 13, 14 are not shown in FIGS. 2A and 2B but they may be comprised in the embodiment of FIG. 3.

According to the embodiment of FIG. 3, the step of inter and / or intra personal homogenizing 21 may comprise the step of standardizing 31 of the at least one physiological signal 15′, the at least one standardized physiological signal 31′ being the input data to the trained machine learning.

By standardizing, we mean putting different variables on the same scale, in other words, a scaling technique where the values of the at least one physiological signal are centered around a mean with a low standard deviation (such as 1 or a unit standard deviation). This means that the mean of the attribute becomes zero and the resultant distribution has a unit standard deviation.

This embodiment may be a way to take into account the variability between different patient.

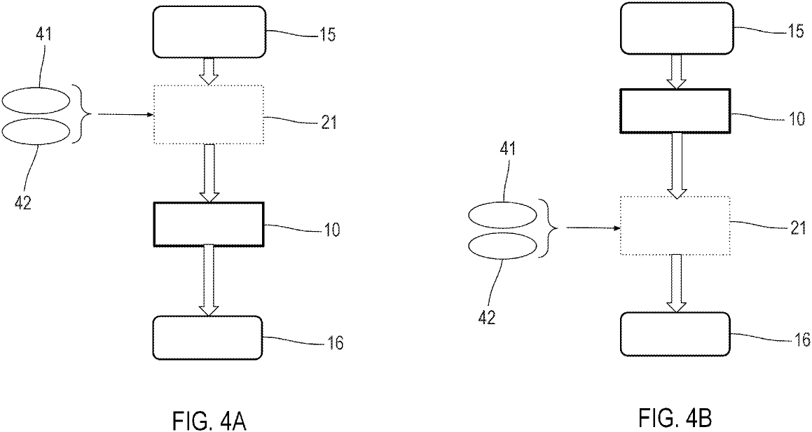

The FIGS. 4A and 4B are two flow charts representing an embodiment of the method for determining a level of certainty of a patient’s response to a stimulus perception of a subjective medical test according to the present description.

As in FIG. 1, the references 15, 10, 16 are the same. The steps and elements 11, 12, 13, 14 are not shown in FIGS. 2A and 2B but they may be comprised in the embodiment of FIGS. 4A and 4B.

According to the embodiment of FIGS. 4A and 4B, the step of inter and / or intra personal homogenizing 21 may comprise a step of detecting at least one reference physiological signal 41 associated to a reference level of certainty 42 of the patient’s response. The at least one reference physiological signal 41 and the reference level of certainty 42 of the patient’s response are a set of reference data. The level of certainty of the patient’s response to the stimulus perception is determined from the at least one physiological signal and from the at the set of reference data.

Thus, with the detected set of reference data at the beginning of the test on the patient and by taking into account these reference data either at the input of the model (in addition to the usual features), or to “threshold” the output according to the “normal” level of the patient, it is a way to know how to manage the differences between people because emotion is not expressed in a standardized way.

This embodiment presents the advantage to personalize the determination of the level of certainty according to the patient and his features at the moment of the test. Further, it allows enriching the input data to improve the training of the machine learning model.

In other words, it’s a kind of calibration per patient which could be done at the start, middle or end of the test to overcome inter-person variability (cultural differences, etc.) and intra-person (current mood, fatigue, etc.).

The reference level of certainty of the patient’s response may be used as an input to the trained machine learning model.

The reference level of certainty of the patient’s response may be used to threshold the output data.

According to further embodiments which can be considered alone or in combination, the physiological signals comprise signals having different modalities and the method comprises formatting the physiological signals having different modalities. Thus, it is possible to use different kinds of physiological signals such as video, audio, text along with micro-expression, pressure measurement, temperature measurement....

For this embodiment, the first step may be to extract unimodal features from each signal for example from a video. Thus, for example, textual, audio and visual features may be extracted.

For extracting visual features from the videos, as explained before, CNN may be used.

For extracting audio features from the videos, OpenSMile may be used or any kind of extraction of sound.

Then, the features from individual modalities may be fused to map them into a joint space thanks to fusion techniques for example concatenation techniques.

The machine learning model or the trained machine learning model may do the formatting.

The formatting may be done just after detecting the at least physiological signal independently of the machine learning model.

According to further embodiments which can be considered alone or in combination, the output and/or the input data is processed.

Further, the present disclosure describes a method for a subjective medical test, which comprises
determining the level of certainty according to the present disclosure, and
informing of the determined level of certainty, and/or
weighting a result of the subjective medical test and/or
changing manually or automatically the stimulus perception according to the determined level of certainty.

Subjective medical test may be: the entire test (e.g. determination of the correction), part of the test (e.g. determination of the OD sphere), or a sub-part of the test (e.g. a step in the determination of the DO sphere).

Further, the present disclosure describes a medical device for a subjective medical test of a patient based on the assessing of the level of certainty of the patient by machine learning, comprising:
a control unit configured to determine the level of certainty of a patient’s response to a stimulus perception of the subjective medical test
the level of certainty being determining from at least one physiological signal of the patient while the patient is providing the response to the stimulus perception,
the at least one physiological signal being as an input data to a trained machine learning model, the determined level of certainty being as an output of the trained machine learning model.

According to an embodiment, the device comprises further
a test unit configured to provide a subjective test associated to stimulus perceptions,
a detector configured to detect at least one physiological signal from the patient while the patient is providing a response to the stimulus perception.

The control unit is configured to determine the level of certainty of the patient’s response to the stimulus perception from the at least one physiological signal according to the method of the present disclosure and according to the embodiment of the method of the present disclosure.

The control unit may be located at the same place than the one of the subjective medical test or at a different place than the one subjective medical test.

The control unit may be located at the same place than the one of the patient or at a different place than the one of the patient.

It presents the advantage to be used for telemedicine / tele optometry or to be used in face to face with the practitioner.

The detector may at least one microphone and/or at least one camera and/ or at least one pressure detector (pressure on an object or blood pressure) and/or at least one temperature detector, electroencephalogram, electroretinogram, cardiac frequency detector or any detector which can measure a physiological signal of the patient.

The detector may be connected to the control unit by a bluetooth connection or a wifi connection.

The detector may be placed on the medical device, on the control unit, on the patient or fix in the room where the test is realized.

The subjective medical test of the medical device may be an ophthalmic test associated to a stimulus perception may be a visual stimulus perception.

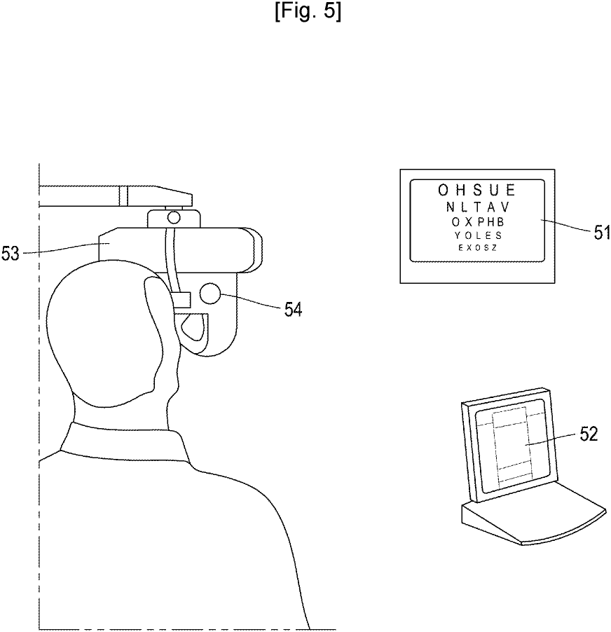

FIG. 5 illustrates a device according to an embodiment of the invention for an ophthalmic test. In particular, FIG. 5 shows the context for using a phoropter head 53 for determining refractive properties or refractive correction need of an eye of a subject who is a wearer of corrective eyeglasses or contact lenses whose correction needs are to be assessed. The phoropter head 53 is mounted on a holder which is further linked to a hinged arm. The hinged arm is further attached to a stationary portion of the phoropter. When assessing the correction needs of the patient, said patient is seated in a seat, and the eyepieces of the phoropter head 53 are placed in front of the patient’s eyes. The patient’s correction needs are evaluated based on the aptitude of the patient to identify the characters displayed on an optotype 51 when he looks through the optical systems arranged behind the eyepieces. The eyepiece and the optotype are the test unit. In this example, the detector 54 is fixed on the phoropter head 53. The control unit 52 is configured to determine the level of certainty of the patient’s response to the stimulus perception from the at least one physiological signal recorded by the detector and also to control the phoropter.

The device according to the present disclosure may be used for any subjective test, more especially when at least some of its steps are automated / calculated. For patient vision evaluation, patient hearing evaluation, patient pain evaluation, patient/client comfort evaluation, ....

For example, for the subjective refraction determination, the method may be used for:
Tests where the patient needs to read letters on a screen (such as acuity): according to her/his ease in reading letters of the line, the practitioner will know which next letter size or which lens should be place in front of the eye of the patient, should be displayed and tested (rather than just taking the same resizing factor at each step); or
Tests where the practitioner would like to perform a final check of the obtained refraction result (verification): the patient expresses its feelings regarding the proposed correction. To avoid to overcorrect the patient, he may have a high level of certainty to be sure that the -+ 0.25 D really improves, otherwise it is kept to the original refraction result.

Many further modifications and variations will suggest themselves to those skilled in the art upon making reference to the foregoing illustrative embodiments, which are given by way of example only and which are not intended to limit the scope of the invention, that being determined solely by the appended claims.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention as defined in the set of claims.

1. A computer implemented method for determining a level of certainty of a patient’s response to a stimulus perception of a subjective medical test, the method comprising:
detecting at least one physiological signal from the patient while the patient is providing a response to the stimulus perception; and
determining the level of certainty of the patient’s response to the stimulus perception from the at least one physiological signal,
the at least one physiological signal being an input data to a machine learning model trained based on a set of training data,
the set of training data including at least one physiological signal associated to a level of certainty of a patient’s response, and
the determined level of certainty being the output of the trained machine learning model.

2. The computer implemented method according to claim 1, further comprising inter and / or intra personal homogenizing the input data or the output of the trained machine learning.

3. The computer implemented method according to claim 2, wherein the inter and/or intra personal homogenizing further comprises standardizing the at least one physiological signal, the at least one standardized physiological signal being the input data to the trained machine learning.

4. The computer implemented method according to claim 2, wherein the inter and/or intra personal homogenizing further comprises :
detecting at least one reference physiological signal associated to a reference level of certainty of the patient’s response, the at least one reference physiological signal and the reference level of certainty of the patient’s response being a set of reference data, and
wherein the level of certainty of the patient’s response to the stimulus perception is determined from the at least one physiological signal and from the set of reference data.

5. The computer implemented method according to claim 4, wherein the at least one reference physiological signal is an input data to the trained machine learning model.

6. The computer implemented method according to claim 4, wherein the at least one reference physiological signal is used to threshold the output data.

7. The computer implemented method according to claim 1, wherein the physiological signals comprise signals having different modalities, and
wherein the method further comprises formatting the physiological signals having different modalities.

8. The computer implemented method according to claim 1, wherein the level of certainty is a category, and
wherein determining the category of certainty further comprises classifying the input data by way of the trained machine learning model to determine the level of certainty.

9. The computer implemented method according to claim 1,
wherein the level of certainty is a score, and
wherein determining the score of certainty further comprises regressing the input data by way of the trained machine learning model to determine the level of certainty.

10. The computer implemented method according to claim 1, wherein the subjective medical test is a subjective ophthalmic test, the stimulus perception is a visual stimulus perception.

11. A computer implemented method for a subjective medical test, comprising:
determining a level of certainty according to claim 1 ; and
informing of the determined level of certainty, and/or weighting a result of the subjective medical test, and/or changing manually or automatically the stimulus perception by taking into account the determined level of certainty.

12. A device for a subjective medical test of a patient, comprising:
control circuitry configured to determine the a level of certainty of a patient’s response to a stimulus perception of the subjective medical test,
the level of certainty being determining from at least one physiological signal of the patient while the patient is providing the response to the stimulus perception,
the at least one physiological signal being as an input data to a trained machine learning model, and
the determined level of certainty being as an output of the trained machine learning model.

13. The device according to claim 12, further comprising
test circuitry configured to provide a subjective test associated to stimulus perceptions, and
a detector configured to detect at least one physiological signal from the patient while the patient is providing a response to the stimulus perception.

14. The device according to claim 13, wherein the detector is at least one microphone and/or at least one camera and/or at least one pressure detector and/or at least one temperature detector.

15. The device according to claim 12,
wherein the subjective medical test is an ophthalmic test, and
wherein the stimulus perception is a visual stimulus perception.

16. The computer implemented method according to claim 3, wherein the inter and/or intra personal homogenizing further comprises:
detecting at least one reference physiological signal associated to a reference level of certainty of the patient’s response, the at least one reference physiological signal and the reference level of certainty of the patient’s response being a set of reference data, and
wherein the level of certainty of the patient’s response to the stimulus perception is determined from the at least one physiological signal and from the set of reference data.

17. The computer implemented method according to claim 5, wherein the at least one reference physiological signal is used to threshold the output data.

18. The device according to claim 13,
wherein the subjective medical test is an ophthalmic test, and
wherein the stimulus perception is a visual stimulus perception.

19. The device according to claim 14,
wherein the subjective medical test is an ophthalmic test, and
wherein the stimulus perception is a visual stimulus perception.
